(19) United States
(12) Patent Application Publication
(10) Pub. No.: US 2008/0234593 A1
(43) Pub. Date: Sep. 25, 2008

(54) Method And Apparatus For Detecting Heartbeats

(76) Inventor: Gerard A. Barone, Orlando, FL (US)

Correspondence Address: BRACEWELL & GIULIANI LLP, P.O. BOX 61389, HOUSTON, TX, 77208-1389, US

(21) Appl. No.: 12/098,792

(22) Filed: Apr. 7, 2008

Related U.S. Application Data
(62) Division of application No. 11/212,859, filed on Aug. 26, 2005.
(60) Provisional application No. 60/604,907, filed on Aug. 27, 2004.

(51) Int. Cl. A61B 5/04; A61B 5/02; A61B 19/00

(52) U.S. Cl. 600/509; 600/508; 128/898

(57) ABSTRACT

A security system for detecting heartbeats. The system monitors vibrations and detects in these vibrations signals representative of a heartbeat based on a library of heartbeat signatures. The system library may have signatures representing multiple heartbeat patterns such that the system can detect the heartbeat of a human or other animal.

### RELATED APPLICATIONS

This application is a divisional of U.S. application Ser. No. 11/212,859 filed Aug. 26, 2005 which claims priority under 35 U.S.C. § 119(e) to U.S. Provisional Application Ser. No. 60/604,907, entitled “METHOD AND APPARATUS TO DETECT EVENT SIGNATURES,” filed on Aug. 27, 2004.

### BACKGROUND OF THE INVENTION

1. Field of the Invention

This invention relates generally to signal processing and more particularly to systems that detect heartbeats.

2. Background

The possibility of detecting concealed people through their heartbeats has been considered. GeoVox Security, Inc. of Houston, Tex. sells an Avion heartbeat detector for security applications. Such detectors operate on the principle that a beating heart creates mechanical shock pulses as it pumps blood through a body. The shock pulses produce vibrations that propagate through the body and through objects in contact with the body.

The vibrations, have a very small amplitude—a fraction of the width of a human hair. Nonetheless, sensors exist that can detect such small vibrations. For example, geophones are used in oil exploration. Geophones are sensitive enough to detect vibrations that emanate from a mechanical device and travel long distances through the earth.

A difficulty in using such small amplitude signals in security applications is that there are many other sources of similar signals with a similar or greater magnitude. For purposes of detecting a signal from a beating heart, these signals are noise. A detection system is likely to mistake the noise for a signal representing a beating heart, creating a “false alarm.”

Detection systems are designed so as not to respond to noise and therefore lower their false alarm rates. However, many methods that a system could use to reject noise reduce the sensitivity of the system to a signal the system needs to detect. Reducing the sensitivity to the signal to be detected is also undesirable because it reduces the chances that the desired signal will be missed, creating a false positive. Both false positives and false alarms are undesirable.

Accordingly, it is desirable for a detection system to have a low false alarm rate while simultaneously providing a low rate of false positives. It would be highly desirable to provide an improved system for detecting heartbeats with a low false alarm rate while simultaneously providing a low rate of false positives.

### SUMMARY OF TIE INVENTION

The invention relates to a method of detecting a heartbeat. The method includes providing a plurality of signatures, each signature being a transformation of a representation of a heart beating. A received vibration signal is processed by, for each of a plurality of portions of the vibration signal, transforming the portion to form a transformed portion. Each of the plurality of transformed portions is compared to the plurality of signatures to detect whether the vibration signal contains one of the plurality of signatures.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a sketch of a prior art system detecting a concealed person.

FIG. 2 is a sketch illustrating a heartbeat pattern, as is known in the art.

FIG. 3 is a block diagram illustrating the creation of a library of signatures.

FIG. 4 is a sketch illustrating the formation of windowed signals from a received signal.

FIG. 5 is a block diagram illustrating processing of windowed signals.

FIG. 6 is a block diagram of an alternative embodiment for processing of windowed signals.

FIG. 7 is a block diagram illustrating in greater detail the detector 660 of FIG. 6.

FIG. 8 is a flowchart of a method of detecting heartbeats.

### DETAILED DESCRIPTION OF THE PREFERRED EMBODIMENTS

A system that detects heartbeat signals by comparing detected signals to signatures in a library is described below. Each signature may represent a different heartbeat pattern. In a preferred embodiment, the library contains signatures representative of the range of heartbeat patterns for a human or other animal that is to be detected. In some embodiments, the library signatures are derived by transforming signals representative of heartbeat patterns by a transfer function representative of structures between the beating heart and sensors.

In further embodiments, the heartbeat signals are processed using one or more frequency domain transforms. In one embodiment, the received signal is processed through a frequency domain transform before being compared to signatures in the library. Tn this embodiment, the signatures in the library are preferably created using the same transform. In another embodiment, the frequency domain transform is performed on the result of the comparison between the received signal and signatures in the library.

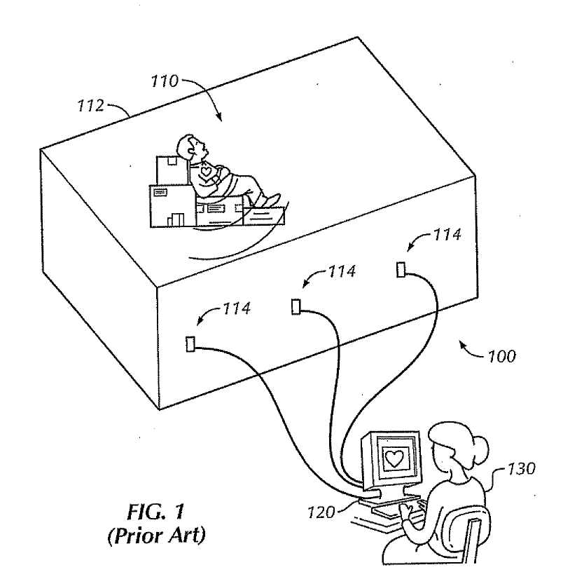

FIG. 1 shows a system 100 in which a heartbeat detection is used for detecting a person concealed in an enclosed space. In the illustrated embodiment, the system is used in connection with containerized cargo. System 100 detects the heartbeat of a concealed person 110 or other animal inside a cargo container 112. Such a system may be used to screen cargo being loaded or unloaded on vehicles such as airplanes or ships. Such a system may detect stowaways, illegal aliens or other people attempting to travel unobserved.

The system employs multiple sensors 114. Sensors 114 might be geophones, microaccelerometers or other similar sensors designed to detect very small vibrations.

The outputs of the sensors 114 are provided to a computer 120. Here, computer 120 acts as both a data processing station and an operator interface station. Computer 120 receives data from sensors 114 and processes it to detect signals representative of vibrations caused by a beating heart from someone within container 112. Computer 120 also provides a user interface for the results of this analysis so that a human user 130 may observe the results and take the appropriate action in response.

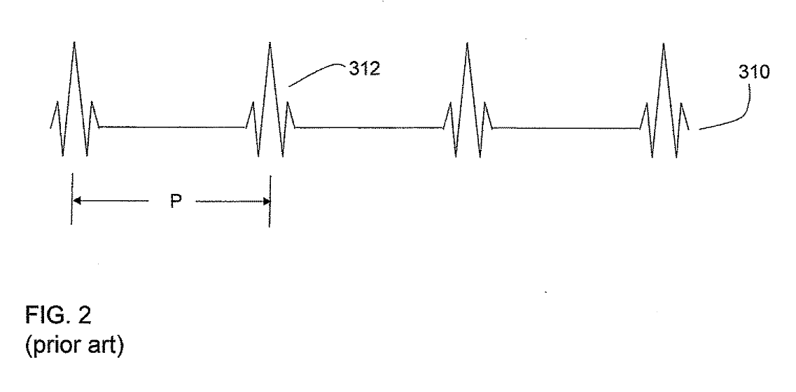

Signals received by the sensor, regardless of the specific application in which the system is used, may be processed to detect signatures representative of a beating heart. One example of processing that may be performed on the received signals is given in FIG. 2. FIG. 2 illustrates a waveform 310 that represents pressure waves launched by a beating heart. The illustration shows shock pulses 312. These pulses are generally periodic, occurring with a period P. The beat pattern of a heart is known in the art and is sometimes referred to as a “balistocardiogram.”

While all beating hearts generally follow the pattern shown in FIG. 2, there can be a wide variation in beat patterns. For example, the heart of an average healthy person beats approximately 60 times per minute. However, heart rates between 40 and 120 beats per minute are not unusual.

To detect such a wide range of possible signals, a system may be created that includes a library of signatures characteristic of a beating heart. A received signal is compared to the library to detect signals indicative of a beating heart. A system such as is shown in FIG. 1 may be modified to employ such a library. In the described embodiment, the modifications are implemented with data processing software in a computer, such as computer 120. However, digital signal processors or other hardware elements may be used to provide the described functions.

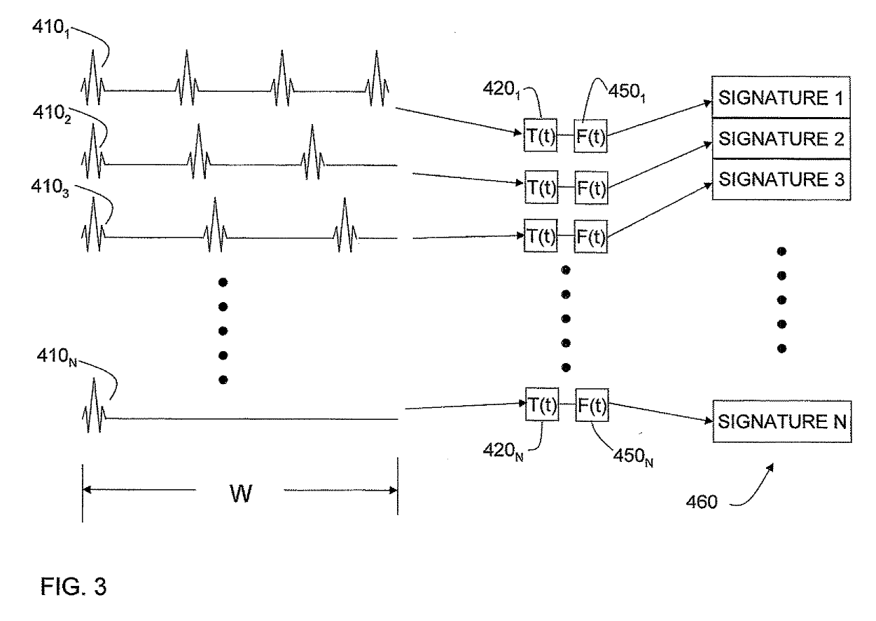

FIG. 3 illustrates the process of forming a library 460 of signatures. The process begins with a collection of heartbeat patterns 410_{1 . . .} 410_{N}. The heartbeat patterns are representative of heartbeat patterns to be detected. For example, if the system is intended to detect concealed humans, the heartbeat patterns should be those of humans. In contrast, if the system is to detect livestock, the patterns 410_{1 . . .} 410_{N} representative of the heartbeat patterns of livestock.

Regardless of the specific type of animal to be detected, the set of patterns 410_{1 . . .} 410_{N} preferably represents the full range of heartbeat patterns that may be encountered. For example, if a human heart may beat between 40 and 120 beats per minute, patterns in the set should represent a heart beating in this range. For example, beat pattern 410₁ represents a quickly beating heart and beat pattern 410_{N} represents a slowly beating heart. The other patterns span the range between these extremes.

The number of beat patterns used to create signatures in the library can be varied to reduce the false alarm and false positive rate of the system. However, as more beat patterns are added, the processing requirements of the system increase, such that the number of beat patterns used to create the library cannot be increased arbitrarily. In the described embodiment, on the order of 100 beat patterns are used. More preferably, about 160 beat patterns are used.

In the illustrated embodiment, the amplitude of all of the beat patterns 410_{1 . . .} 410_{N} has been normalized. Alternatively, the beat patterns may be normalized in other ways, such as to have the same energy. Also, FIG. 3 shows that the beat patterns 410_{1 . . .} 410_{N} in the set vary in beat frequency. If the signals to be detected might vary in other parameters, the set used to create the library preferably contains members representative of the range of variations of every parameter and combination of parameters.

The beat patterns 410_{1 . . .} 410_{N} might be obtained empirically by collecting multiple examples of the types of signal to be detected. Alternatively, the beat patterns might be generated through computer modeling and simulation. For example, a shock pulse 312 might be modeled and the set of beat patterns generated by repeating this shock pulse at different periods.

In the embodiment pictured in FIG. 3, each of the beat patterns 410_{1 . . .} 410_{N} has a duration, W. Though a heartbeat pattern might repeat for a very long time, a window in time, denoted W, is selected for processing. In the illustrated embodiment, W is on the order of seconds, preferably between 1 and 5 seconds. W is preferably about as long as the period P of the beat pattern with the lowest beat frequency.

FIG. 3 pictures the beat patterns 410_{1 . . .} 410_{N} as continuous signals. However, the described embodiment is implemented through computer data processing. Therefore, all signals are preferably in digital form. If beat patterns 410_{1 . . .} 410_{N} are generated by computer, they will be in digital form. If they are generated from empirical data, they may be in analog form, but can be converted to digital form before farther processing using known analog data capture techniques or any other suitable method.

To convert each beat pattern to a signature, the beat pattern is processed by a transfer function T(t). The transfer function represents the effect of the environment on a shockwave generated by a beating heart. For example, in the system illustrated in FIG. 1, transfer function T(t) represents the changes induced in the shockwave generated by the heart of person 110 as the shock wave propagate through the human body and container 112 to sensors 114.

The transfer function T(t) may be obtained by modeling the components of the environment. Known modeling techniques may be used. The transfer function may alternatively be obtained empirically by establishing conditions representative of the conditions under which signals will be detected. For example, an impulse or other signal may be applied and the result measured. Known signal processing techniques can be used to derive the transfer function of a system by measuring the response of the system to a known input. If the container is on a truck with a suspension system, or an elevator suspended on a cable or the signal path includes other mechanically active elements, using a transfer function to compute heartbeat signatures may increase the overall accuracy with which heartbeats may be detected. In other embodiments, such as when a container is sitting on the ground, stacked on other containers, or the sensors are mounted directly to the container, the transfer function may be approximate 1 or may be unknown and processing with a transfer function may be omitted.

Where data is to be gathered empirically, signals in the form of the beat patterns 410_{1 . . .} 410_{N} could be used as stimulus signals. If this scenario can be created, it is not necessary that the transfer function be ascertained. Rather, the measured value in response to each of the beat patterns 410_{1 . . .} 410_{N} would represent the output of one of the transfer function blocks, such as 410_{1 . . .} 410_{N}, which are signals required for the next step in processing. However, separately generating the transfer function and representative beat patterns allows new entries to be readily added to the library 460 if it is determined that more beat patterns are needed to accurately represent the full range of beat patterns or if it is determined that the library needs to be regenerated with a different transfer function.

In the next step of the processing, the signals are transformed according to a transform F(t). As will be described below, a received signal is transformed before comparison to the signatures in the library. In such an embodiment, it is preferable that the beat patterns be similarly transformed before being stored in library 460. In a described embodiment, F(t) represents a frequency domain transform. A known frequency domain transform may be used. For example, a Discrete Fourier Transform (DFT) may be used. However, other transforms may be used, such as the discrete cosine transform. Alternatively, a wavelet transform may be used.

Further, it is possible that a signal may be subject to multiple transforms before being stored in library 460. The transforms may be applied sequentially or separately. Where transforms are applied separately, library 460 may contain multiple entries for each of the beat patterns 410_{1 . . .} 410_{N,} with an entry for each beat pattern transformed with each of the transforms.

The transformed beat patterns are preferably stored in library 460 in advance of use of the system. Preferably, representative patterns are selected and a library is created as the system is developed. However, it is possible that the library is built adaptively. As items are inspected, data may be gathered to generate new signatures.

Where the types of objects to be inspected vary so widely that some will have substantially different transfer functions, library entries may be generated using each transfer function. For example, where a system may be used to process signals from either large or small containers, a transfer function may be generated for each type of container. Each of the beat patterns 410_{1 . . .} 410_{N} would then result in two entries in library 460, one computed with each transfer function.

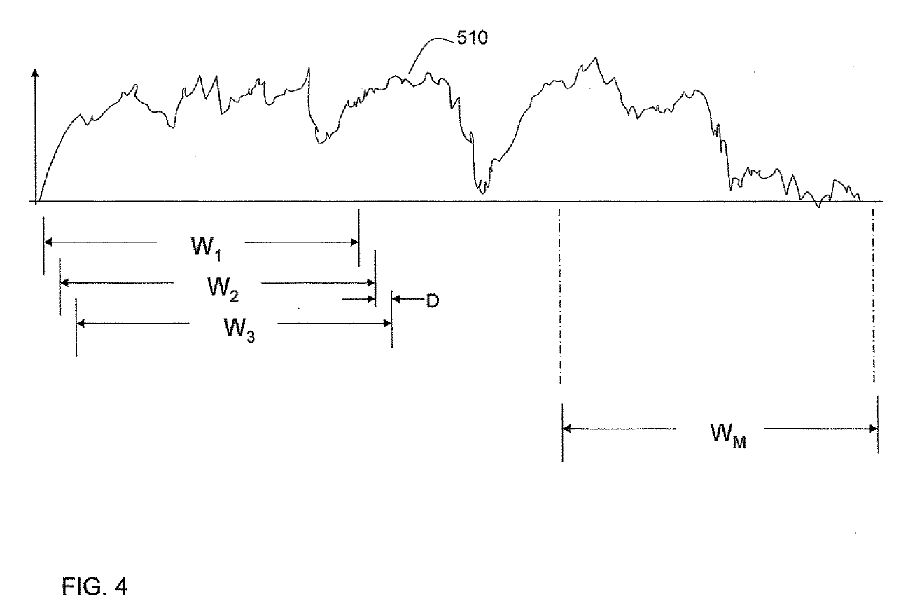

Once the library 460 is developed, the system may be deployed and used. FIG. 4 shows a signal 510, such as may be received by one of the sensors 114. Signal 510 does not contain a recognizable heartbeat signal. A heartbeat signal may, however, be present and simply masked by noise. Processing as described below will be performed on signal 510 to detect a heartbeat signal.

As will be described below, signal 510 maybe processed in windows. FIG. 4 shows a series of windows, W_{1 . . .} W_{M}. Each window preferably has a duration W, which is the duration of signals used to generate signatures in library 460. Further, the windows are spaced in time by an amount D. Preferably, W will be on the order of seconds and D will be a fraction of W, preferably on the order of 10's of milliseconds. By dividing signal 510 in this fashion, M separate but overlapping window signals are created for processing. In one embodiment, signal 510 is collected over a duration of approximately six seconds and each window has a duration of approximately four seconds.

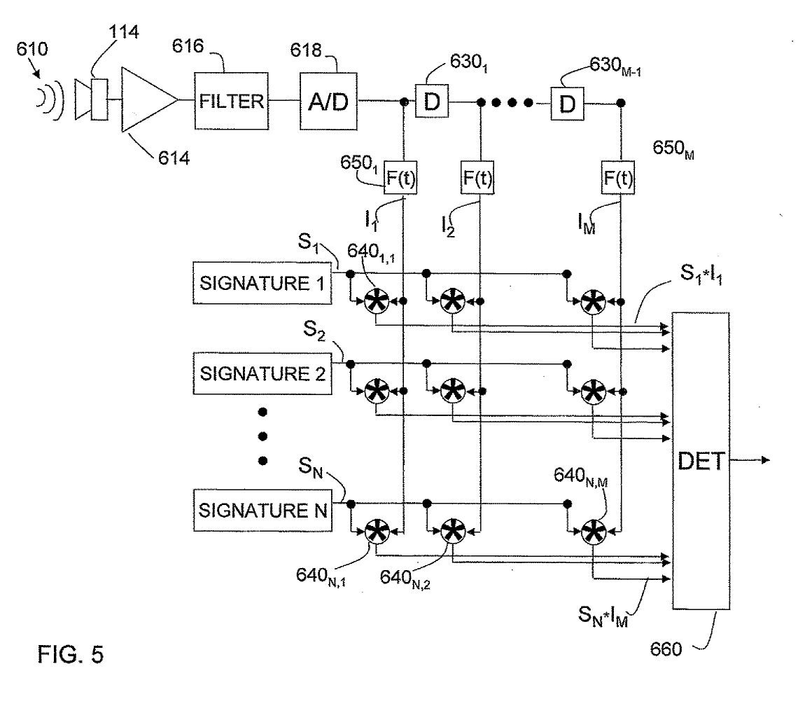

FIG. 5 shows in block diagram form processing that may be used to detect heartbeats in signal 510. As shown, vibrations 610 impinge on a sensor 114, which produces an electrical signal. The output of sensor 114 is amplified by amplifier 614 and then filtered in filter 616. Amplifier 414 may be a high gain, low noise instrumentation amplifier as known in the art. Filter 616 may be a signal conditioning filter as known in the art. The resulting conditioned signal is converted to digital form in A/D converter 618. As described above, processing is preferably performed in digital form, but comparable processing operations could be performed on analog signals. If analog processing is desired, AD converter 618 could be omitted.

The digital signal is provided to a chain of delay elements 630_{1 . . .} 630_{M-1}. Each delay element provides a delay, D. In this way, the output of each delay element 630_{1 . . .} 630_{M-1} forms the signal in one of the windows W_{1 . . .} W_{M} (FIG. 4). The undelayed signal forms the signal in the first window.

In the embodiment shown in FIG. 5, each of the windowed signals is transformed as illustrated at 630_{1 . . .} 630_{M-1}. Here, a frequency domain transformation is used. In the described embodiment, the transformation is the same transformation used to form the library of signatures. In one contemplated embodiment, two transforms are used—a DFT and a wavelet transform.

The transformed windowed signals are denoted I_{1 . . .} I_{M}. Each of the transformed windowed signals is provided to a multiplier array, made up of multipliers 640_{1,1 . . .} 640_{N,M}. Each multiplier multiplies one of the transformed window signals I_{1 . . .} I_{M} by one of the signatures S_{1 . . .} S_{N} in the library 460 (FIG. 3). In the described embodiment, each of the windowed signals and each of the signatures in the library span a time window of duration W and have the same number of values within that window. Accordingly, there is a point-for-point correspondence between values of the signals I_{1 . . .} I_{M} and values in the signatures S_{1 . . .} S_{N}. The two signals may be multiplied by multiplying the successive values of the signals point-by-point.

If one of the transformed window signals I_{1 . . .} I_{M} is similar to one of the signatures, S_{1 . . .} S_{N}, the two signals should have similar frequency spectra. Thus, the high points of each will align and when the two signals are multiplied, the product signal will have high values corresponding to those high points. In contrast, when a windowed signal does not correspond to a signature, the frequency spectra of the signals will be different and there will be fewer points where the high points of those signals align. As a result, the product signal will contain fewer high points and, the high points are less likely to be as large.

As described above, the received signal 510 is divided into multiple, overlapping windows. Preferably, the width and amount of overlap of the windows is selected to ensure that, if signal 510 contains a heartbeat signal, that signal will be aligned in one of the windows W_{1 . . .} W_{M} in a way that aligns with a signature in library 460. Thus, all of the transformed window signals I_{1 . . .} I_{M} are preferably multiplied by each of the signatures S_{1 . . .} S_{N}. These products S₁*I_{1 . . .} S_{N}*I_{M} are all examined to see which, if any, have values indicating that the window signal matches a signature. These signals are passed to detector 660 for this analysis.

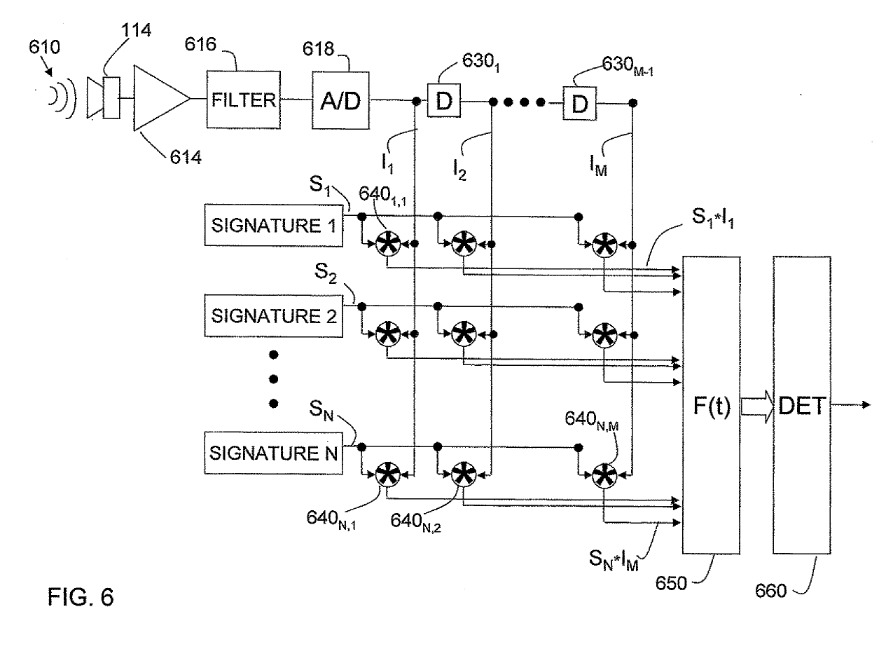

FIG. 6 shows an alternative embodiment of the process for comparing a received signal to signatures in a library. In this embodiment, the windowed signals are multiplied by signatures in the library before they are frequency domain transformed. For this embodiment, the signatures in the library are preferably still time domain signals. Thus, the transformation process at 450_{1 . . .} 450_{N} (FIG. 3) may be omitted in creating the library of signatures for use in this embodiment. However, as in the embodiment of FIG. 5, the received signal is divided into window signals, each of which is multiplied by each of the signatures.

The product signals are then frequency domain transformed, as illustrated at 650. In the illustrated embodiment, each product signal is separately transformed. As described above, one or more known frequency domain transforms may be used. In one contemplated embodiment, a DFT and a wavelet transform are both used, with the transforms being provided in parallel such that each product signal results in two transformed signals. The transformed product signals are then analyzed to detect which, if any, contain, heartbeat signals. Detection is performed by detector 660.

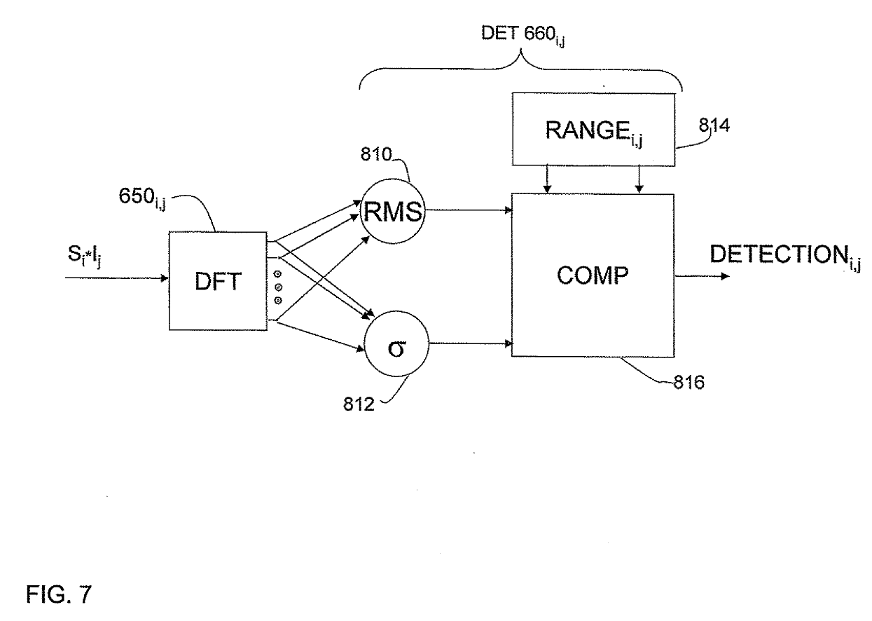

FIG. 7 shows additional details of detector 660. FIG. 7 illustrates the processing of one product signal. Each product signal may be similarly processed. In the embodiment of FIG. 6, the product signals are transformed to the frequency domain before processing by detector 660. Accordingly, FIG. 7 shows a DFT 650_{i,j} formed on the product signal S₁*Iⱼ before application to detector 660. In the embodiment of FIG. 5, a frequency domain transform is performed before the product signal is formed, and DFT 650_{i,j} may be omitted.

The product signal, when transformed to the frequency domain, is a series of frequency values. These values are compared to predetermined criteria to indicate a match between the windowed signal Iⱼ and the signature Sᵢ. A match can be taken as an indication that windowed signal Iⱼ contains a heartbeat in the form 450ᵢ, which was used to generate the signature Sᵢ.

In the described embodiment, the comparison is made using statistical properties of the frequency domain signal. In the illustrated embodiment, these statistical properties are the average value and the variance.

The average value is computed at 810. In the described embodiment, the average is computed according to the Root Mean Square (RMS) method. The variance of the frequency domain values is also computed at 812. Both the RMS and variance are known statistical properties and may be computed in accordance with any known method.

The computed RMS and variance values are provided to comparator 816. Where multiple frequency domain transforms are used, statistical properties of each transformed signal may be computed separately and provided to comparator 816.

Comparator 816 compares the statistical properties of the measured signals to a range or ranges that are indicative of a match. If the computed value for the signal falls within the range, comparator 816 outputs an indication that there is a match.

The predetermined ranges might be the same for all combinations of S₁*I_{1 . . .} S_{N}*I_{M}. Alternatively, each product signal Sᵢ*Iⱼ may have different predetermined ranges. Alternatively, each signature in the library may have different predetermined ranges.

The predetermined ranges may be determined empirically or heuristically. If determined empirically, the values may be computed at the time the system is installed or may be adaptively computed as the system is in operation. Various processes for identifying patterns in data are known and may be employed to set the ranges. For example, data may be collected by applying training signals of known properties and observing the outputs. The outputs may be analyzed to identify the ranges that result when an input signal contains a match to one of the signatures.

As another example, the ranges may be set by computing statistical properties on the frequency properties of the signatures in the library. For example, a match may be determined if the RMS and variance values of the product signal Sᵢ*Iⱼ are within 15% of the RMS and variance values computed for the signature Sᵢ.

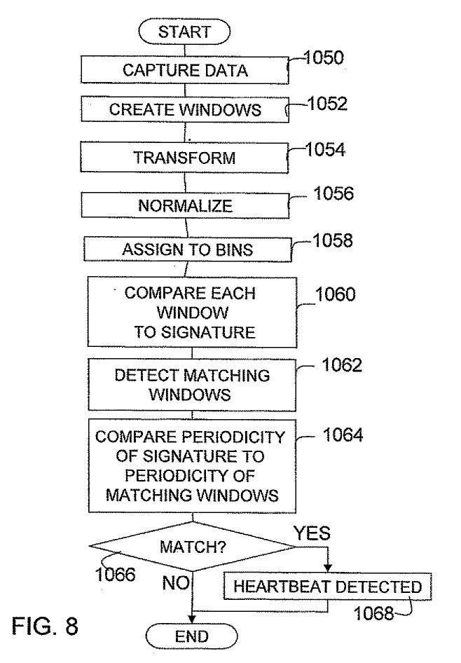

Regardless of the specific method used to set the range, if the statistical properties of the signal are within the range, a detection is indicated. FIG. 8 shows a flowchart of an exemplary process that may be used to detect a heartbeat using a system as described above. The process begins at block 1050 where data is captured. Data from one or more sensors may be captured by sampling the data and converting it to digital form.

The process continues to block 1052. At block 1052, captured data is formed into a plurality of successive and overlapping windowed signals. Each window is preferably large enough to contain a signal representing a heartbeat.

At block 1054, each of the windowed signals is transformed. In the embodiment, illustrated, a frequency domain transform is used. One suitable frequency domain transform is a high-resolution Fourier transform. However, other suitable transforms may be used. The transform performed at block 1054 creates, for each windowed signal, a series of frequency coefficients. Each coefficient represents the frequency content of the windowed signal at a specific frequency.

At block 1056, each of the frequency domain representations of the windowed signal is normalized. In this embodiment, a frequency domain representation is normalized by selecting the largest frequency coefficient. The multiplicative inverse of the largest frequency coefficient is computed and each of the frequency coefficients is multiplied by this inverse value. At the end of the normalization step at block 1056, the largest frequency coefficient in each of the transformed windowed signals will be one and all other coefficients will be normalized to a value less than one.

At block 1058, the normalized frequency coefficients are assigned to bins. Normalizing and assigning to bins facilitates comparison of signals. In one embodiment, five bins are used, having values of 0, 0.4, 0.6, 0.8, and 1.0. Any suitable mapping between the normalized coefficient values and bones may be used. In the illustrated embodiment, normalized coefficients having a value above 0.8 are reset to a value of 1.0. Normalized coefficients with a value above 0.6 and equal to or less than 0.8 are reset to a value of 0.8. Similarly, normalized coefficients above 0.4 and equal to or less than 0.6 are reset to a value of 0.6. Normalized coefficients with values above 0.2 and equal to or less than 0.4 are reset to a value of 0.4. Normalized coefficients with a value of 0.2 or less are reset to equal 0.

With the values of the normalized coefficients mapped to one of a small number of bins, processing continues to block 1060. At block 1060, each of the normalized window signals is compared to a heartbeat signature. For simplicity, comparison to a single signature is described. But, each signal may be compared to more than one signature by repeating the processing in block 1060, 1062, 1064, 1066, and 1068.

At block 1060, the normalized window signals may be compared to a signature in any suitable way, such as the embodiment shown in FIG. 7. As described above in connection with FIG. 7, each of the normalized transformed windowed signals is multiplied on a point-by-point basis with a signature. Each windowed signal matching the signature may be selected at block 1062 for further processing.

In one embodiment, matching signals may be selected by computing the average and standard deviation of these point-by-point multiplications. The average and standard deviation may be compared to a predetermined range or threshold values that signify a match to the signature.

Block 1064 indicates a processing step that may be employed for heartbeat signatures. For example, if a heart beats every half second, it may be expected that the signature of a heartbeat will appear in windowed signals representing portions of the original signal spaced apart by half-second intervals. Therefore, at block 1064, the periodicity of the signature is compared to the periodicity of the windows at which a match occurs.

If there is a high level of correlation between the periodicity of the signature and the periodicity of matching windows, processing at block 1066 indicates that a heartbeat is detected. If a heartbeat is detected, processing continues to block 1068. At block 1068, action appropriate for a detected heartbeat may be taken, such as storing an indication of the heartbeat in memory. Conversely, if there is no match between the periodicity of the signature and the periodicity of matching windows, the process of FIG. 8 may end without an indication that a heartbeat has been detected. Basing a detection of a heartbeat on the periodicity of the signature and periodicity of the matching windows increases the confidence with which a heartbeat is detected and therefore reduces the false alarm rate of the system. In some embodiments, the processing is represented by block 1064, 1066 and 1068 may be omitted such that a heartbeat is reported as detected when any of the windowed signals matches a heartbeat signature.

Having described embodiments of the invention, one of skill in the art will appreciate that multiple alternative embodiments might be created.

For example, FIG. 1 shows a single computer 120. Generally, data may be collected, processed, and output by one or more processors in any suitable configuration, which could be a single computer or multiple computers interconnected by a network. For example, an embodiment was described as being implemented in software programmed on a computer work station, which might be a standard desk top computer. A more sophisticated computer, including multi-processor work stations might be employed. Further, array processors and dedicated signal processing hardware, including Application Specific Integrated Circuits (ASICs) may be used to implement the described processes.

As another example of variation, the described embodiments include delay elements to produce window signals. If the system is not implemented to perform real time processing, physical elements introducing delay into a data stream may not be required. The delay may be introduced by storing the entire signals and retrieving the desired portions when needed.

As another example, it is described that the received signal is divided into multiple overlapping windows so that a heartbeat signal in the received signal will appear in one of the windows with the same alignment as the signal used to create a signature in the library. A similar effect may be achieved in other ways. For example, the library could contain multiple signatures for each of the heartbeat patterns 410_{1 . . .} 410_{N} with entries derived by shifting each heartbeat pattern by an amount D before forming the signature. Alternatively, the signatures in the library may be time shifted to generate multiple signals before use instead of or in addition to forming overlapping windows for the received signal as shown in FIG. 4.

As a further example of possible variations, the orders of various operations might be reversed. For example, FIG. 3 shows a transfer function followed by a frequency domain transform. The order of these operations might be reversed by representing the transfer function in the frequency domain. As another example, FIG. 3 shows that the transfer function is applied before signatures are stored. The signatures could be stored without applying the transfer function and the transfer function could be applied as the signatures are retrieved from the library. Such embodiment may be useful, for example, if the transfer function may be different for different items under inspection. For example, if multiple types of containers are to be inspected, it might be desirable to select the transfer function appropriate for the specific container under inspection.

Similarly, FIG. 5 shows a transform being applied to each delayed signal before it is multiplied by a signature library 460. In this embodiment, a frequency domain transform is applied to each signature before it is stored in the library. FIG. 6 shows alternative processing in which the transform 650 is applied after each delayed signal is multiplied by a signature. In this embodiment, signatures in the library may not be transformed at all.

Also, it was described that a windowed signal and a signature are compared by a point to point multiplication. This multiplication might be viewed as a form of convolution. Other forms of convolution might be used. Related functions, such as a correlation function might be used to compare the signals to the signatures.

Further, while the DFT and wavelet transform are described, other transforms might be used.

Moreover, the statistical properties used to ascertain whether a windowed signal matches a signature are illustrative. Other types of averages or other functions that indicate the distribution of values might be used.

As a further example, each window signal was described to be processed independently. Additional information may be obtained by further processing, such as by comparing or combining the results of computations on multiple window signals. For example, if a window, Wₓ is determined to contain a signal matching a heartbeat pattern with a period P, a later window W_{x+y} should also match that same heartbeat pattern. Here Y can be computed by dividing the period of repetition of the heartbeat pattern, P, by the spacing D between the windows. The confidence of the detection may be significantly increased if the patterns of windows matching heartbeat patterns are analyzed.

In the described embodiment, the output of each sensor 114 may be independently processed using the above described approach. As another example of post processing that might be employed to improve performance of the system, the outputs of multiple sensors might be compared to ascertain whether a heartbeat was detected in the signal from multiple sensors.

Preferably, the received signal is normalized before processing. The normalization step is not explicitly shown. Preferably, the signal in each window is normalized separately. However, normalization might take place at any convenient place in the processing. For example, amplifier 614 might contain automatic gain control, which would provide a form of normalization.

What is claimed is:

1. A method of detecting a heartbeat, comprising:
a) providing a plurality of signatures, each signature being a transformation of a representation of a heart beating;
b) receiving a vibration signal;
c) for each of a plurality of portions of the vibration signal transforming the portion to form a transformed portion; and
d) comparing each of the plurality of transformed portions to the plurality of signatures to detect whether the vibration signal contains one of the plurality of signatures.

2. The method of claim 1, wherein the act c) comprises performing a frequency domain transform on each of the plurality of portions.

3. The method of claim 2, wherein the act c) comprises performing a Fourier transform on each of the plurality of portions.

4. The method of claim 2, wherein the act c) comprises normalizing the transformed portion.

5. The method of claim 4, wherein each signature and each transformed portion has a plurality of frequency components and the act d) comprises, for each pair of a transformed portion and a signature compared:
a) forming products of the frequency components of the signature and the frequency components of the transformed portion, and
b) computing statistical properties of the products.

6. The method of claim 5, wherein computing statistical properties comprises computing a mean and deviation.

7. The method of claim 5, wherein the act d) additionally comprises comparing the statistical properties to predetermined values.

8. The method of claim 1, wherein the act a) comprises providing a plurality of signatures, representing a heart beating with plurality of different beat patterns.

9. The method of claim 1, used in a method of detecting a person concealed in an enclosed space having a surrounding structure, wherein the act b) of receiving a vibration signal comprises receiving a vibration signal through the surrounding structure.
